(19) United States
(12) Patent Application Publication
(10) Pub. No.: US 2026/0175084 A1
(43) Pub. Date: Jun. 25, 2026

(54) FITNESS DEVICE FOR GENERATING AND PRESENTING PERSONALIZED PACE FEEDBACK BASED ON USER'S FITNESS PROFILE

(71) Applicant: Apple Inc., Cupertino, CA (US)

(72) Inventors: Burak Arda Ozilgen, San Francisco, CA (US); Chin S. Han, San Jose, CA (US)

(21) Appl. No.: 19/428,059

(22) Filed: Dec. 19, 2025

Related U.S. Application Data
(60) Provisional application No. 63/738,486, filed on Dec. 23, 2024.

(51) Int. Cl. A63B 24/00

(52) U.S. Cl. CPC A63B 24/0062; A63B 24/0075; A63B 2024/0065; A63B 2230/755

(57) ABSTRACT

Embodiments are disclosed for a fitness device that generates and presents PACE feedback based on a user's physical fitness profile. In an embodiment, a method comprises: receiving, with at least one processor of a fitness device, nutrition information for a food item to be consumed by a user of the fitness device; determining, with the at least one processor, a fitness profile of the user, including activity data of the user and a fitness goal of the user; generating, with the at least one processor, physical activity calorie equivalent data based on the nutrition information, the physical fitness profile and previous calories consumed by the user; adjusting, with the at least one processor, the exercise goal of the user based on the physical activity calorie equivalent data; and presenting, with the at least one processor, the adjusted exercise goal to the user.

### CROSS-REFERENCE TO RELATED APPLICATION

This application claims priority to U.S. Provisional Patent Application No. 63/738,486, filed Dec. 23, 2024, the entire contents of which are incorporated herein by reference.

### TECHNICAL FIELD

This disclosure relates generally to health monitoring, and more particularly to fitness applications.

### BACKGROUND

Physical Activity Calorie Equivalent (PACE) is a method that is used to inform people of the typical number of minutes of exercise (e.g., walking) that would be required to burn the calories in a certain food item. This method has been used on menus in various parts of the world. One of the major shortcomings of PACE is that it is not personalized because it does not take into consideration a particular individual's fitness goals, metabolism, preferred activity type, activity levels or workout habits.

### SUMMARY

Embodiments are disclosed for a fitness device that generates and presents personalized PACE feedback based on a user's fitness profile.

In some embodiments, a method comprises: receiving, with at least one processor of a fitness device, nutrition information for a food item to be consumed by a user of the fitness device; determining, with the at least one processor, a fitness profile of the user, including activity data of the user and a fitness goal of the user; generating, with the at least one processor, personalized physical activity calorie equivalent data based on the nutrition information, the fitness profile and previous calories consumed by the user; adjusting, with the at least one processor, the exercise goal of the user based on the physical activity calorie equivalent data; and presenting, with the at least one processor, the adjusted exercise goal to the user.

In some embodiments, the fitness profile further includes demographic data for the user.

In some embodiments, the fitness profile further includes historical workout pattern data for the user.

In some embodiments, the fitness profile further includes a preferred exercise type of the user.

In some embodiments, the fitness profile further includes an exercise habit of the user.

In some embodiments, the nutrition information for the food item to be consumed by a user is captured from a nutritional fact label for the food item by a camera of the fitness device.

In some embodiments, the activity data of the user is determined from sensor data provided by sensors of the fitness device.

In some embodiments, the fitness profile is provided by a fitness application running on the fitness device.

In some embodiments, the adjusted exercise goal includes an exercise type for the user to engage in to burn off calories in the food item, and wherein the exercise type is the preferred exercise type in the physical fitness profile.

In some embodiments, the adjusted exercise goal includes an amount of time for the user to engage in the exercise type to burn off the calories in the food item.

In some embodiments, the fitness profile further includes upcoming or scheduled workout information for the user (e.g. through user's text messages with contacts, scheduled calendar events).

Particular embodiments described herein provide one or more of the following advantages. Cameras and other sensors on a smartphone, tablet computer, virtual reality (VR)/augmented reality (AR) goggles or other fitness device generates and presents personalized contextual feedback to a user based on the user's current calorie consumption and fitness profile, including, for example, informing the user regarding the duration, level and type of physical activity needed to burn calories contained in a food item consumed by the user. These embodiments improve on the PACE method which is not personalized for the user.

### BRIEF DESCRIPTION OF THE DRAWINGS

FIG. 1 illustrates a system implemented on a fitness device that generates and presents personalized PACE feedback based on a user's fitness profile, according to one or more embodiments.

FIG. 2 is a flow diagram of a process implemented by a fitness device that generates and presents personalized PACE feedback based on a user's fitness profile, according to one or more embodiments.

FIG. 3 is a block diagram of an architecture for a fitness device that generates and presents personalized PACE feedback based on a user's fitness profile, as described in reference to FIGS. 1-2.

### DETAILED DESCRIPTION

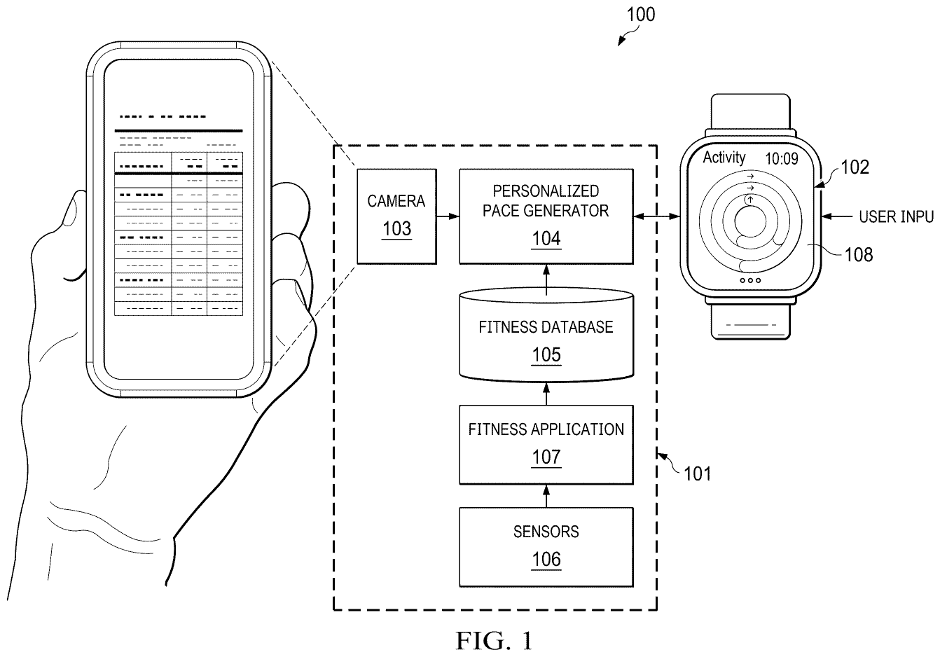

FIG. 1 illustrates system 100 implemented on fitness device 101 (e.g., a smart watch, fitness band, headset, etc.) that generates and presents personalized PACE feedback based on a user's fitness profile, according to one or more embodiments. System 100 includes display 102, camera 103, personalized PACE generator 104, fitness database 105, fitness application 107 and sensors 106 (e.g., accelerometers, gyros, biosensors, etc.). In the example shown, fitness device 101 is a wrist wearing device, such as a smartwatch. However, fitness device 101 can be any device capable of tracking and storing fitness data and determining personalized PACE information (e.g., a smartphone, tablet computer, fitness band, headset).

In an embodiment, a user captures an image of a nutrition label from a food item with camera 103 (e.g., a video camera). A nutrition label typically includes information about the serving size, calories per serving, total fat, saturated fat, trans fat, cholesterol, sodium, total carbohydrates, dietary fiber, sugar, added sugar, protein, and the percentage of daily value for each nutrient listed, allowing consumers to compare the nutritional content of different foods easily.

Fitness application 107 running on at least one processor of the fitness device records various fitness data for the user based on sensor data provided by sensors 106 and generates a fitness profile the user which is stored in fitness database 105. In some embodiments, fitness application 107 app helps a user track their fitness progress, share their fitness activities and access workouts and meditations. For example, fitness application 107 can computes how many calories the user has burned based on sensor data and set a fitness goal for the user (e.g., a move goal). In some embodiments, the user can see their physical activity details and history, trends, and awards for meeting their fitness goals. In some embodiments, fitness application 107 offers a variety of workouts, including strength training, high-intensity interval training (HIIT), core, meditation, yoga, Pilates, dance, kickboxing, cycling, treadmill, and rowing. In some embodiments, the user can create their own program based on their preferences, or choose from programs for beginners, 30-day core challenges, etc. In some embodiments, fitness application 107 allows the user to track their progress towards their fitness goals, view their completed workouts, and receive a workout summary.

In some embodiments, fitness application 107 generates fitness data using sensor data from sensors 106. For example, sensors 106 can include an accelerometer that provides acceleration data that can be used by a digital pedometer to count steps taken by the user, by looking at signatures (e.g., peaks and valleys) in the frequency domain that are indicative of step taking. The step data can be used with other data (e.g., the user's height, weight, gender, and age) to determine calories burned, distance traveled and other fitness data or metrics. Other sensor data, such as barometer data, can be used to determine if the user is walking/running on an incline.

In some embodiments, an energy expenditure model can be used to determine calories consumed, such as a Metabolic Equivalents (MET) model which is based on how much oxygen the body consumes during activity compared to how much oxygen the body consumes at rest. For example, most individuals at rest utilize 3.5 mL of oxygen per kg of body weight per minute, which equates to 1 kcal/kg/hr. When performing exercise, MET values are assigned to various forms of physical activity to determine how many calories are expended during that activity. In other embodiments, more personalized energy expenditure models can be used based on work rate and/or heart rate captured by a heart rate sensor.

In some embodiments, calories burned can be determined as described in U.S. Patent Publication No. 2016/0058356, for “Method and System to Calibrate Fitness Level and Direct Calorie Burn Using Motion, Location Sensing and Heart Rate,” published on Mar. 3, 2016, which is incorporated herein by reference in its entirety.

In some embodiments, personalized PACE data generator 104 receives user input (e.g., height, weight, age, gender) through a graphical user interface (GUI) presented on display 103 of fitness device 101 and fitness data from fitness database 105 and generates personalized PACE data. The personalized PACE data informs the user of, e.g., a typical number of minutes of exercise (typically walking) that would be required to burn the calories in a certain food item. Because the personalized PACE data takes into consideration the user's fitness goals, metabolism, preferred activity type, activity levels or workout habits, it provides superior result to the convention PACE method.

In operation, personalized PACE generator 104 uses camera 103 and the fitness data in fitness database 103 provided by fitness application 107 to inform the user how many minutes of activity they personally would need to burn the calories in a consumed food item based on their activity routine as learned by fitness application 107. For example, take an example of a user who is considering eating a bag of chips. The user picks up the bag of chips and uses camera 103 to capture an image of the nutritional facts label on the food item, including the number of calories pers serving. Personalized PACE generator 104 uses fitness data from fitness database 105 to assess the user's recent workout patterns (e.g. weekly, monthly), or routines such as how much the individual has historically worked out that day of the week, including what type of workout, the user's preferred exercise type, exercise habits and fitness goals.

Based on the fitness data and demographic information (e.g., age, weight, gender, etc.) entered by the user, the user is given personalized contextual feedback on the type and quantity of exercise required to burn off that bag/serving of chips, such as, e.g., the number of minutes for a certain type of workout, or what a recommended portion size would be based on their typical activity levels if they are working towards a fitness goal. For the example case of choosing whether or not to eat a 90-calorie bag of chips, an input for a customized suggestion could include the number of calories in the food being consumed or is contemplating consuming by the user, which can be captured by camera 103 from the nutritional facts label. In some embodiments, the nutritional facts can be obtained through use of a bar code (e.g., QR code) or any other suitable input method including user input through a graphical user interface. In some embodiments, this nutritional fact information could be extracted using an a priori algorithm (e.g. a computer vision approach to read a nutritional label, a computer vision algorithm to identify a food).

In some embodiments, a customized recommendation is presented to the user (e.g., displayed and/or through audio) could consider the users physical fitness profile, which can include the number minutes of exercise the user engaged in for the day and the workout history of the user provided by fitness application 107. Instead of informing the user about their absolute caloric intake, the personalized PACE generator 104 can use the current fitness level of the user (e.g., a scale from 1 to 5 with increasing fitness), including but not limited to how much the user moved up in levels until the present. The personalized PACE generator 104 contextualizes what the user is about to eat, or what they have just eaten, in terms of how much they've moved during that day. For example, the personalized PACE generator 104 can contextualize that the number of calories in the bag of chips is equivalent to 70% of the calories the user burned today, or the number of calories in the bag of chips is equivalent to 35% of the calories the user burned during their morning's outdoor run.

In some embodiments, the user's fitness goals can be adjusted. For example, if the bag of chips is outside of the individual's calorie budget for the day their calorie goal for the day would be a nominal fitness goal calorie target for the day plus a number of calories in the bag of chips consumed. The goal would revert back to the nominal fitness goal calorie target for the day on the next day. In another embodiment, in response to a prompt the user can choose to extend their workout time during a workout session to burn off the calories from the chips they consumed. For example, the user starts a workout, then selects the chips from a drop-down menu of consumed foods. The workout time end goal is determined by the calories in the consumed chips (90 calories in this example), and a workout target notification is sent to the user once their calories burned during the workout is equal to or greater than the calories in the chips they consumed (e.g. “Workout goal accomplished” after hitting 90 calories, or their usual workout goal+90 calories, respectively).

In some embodiments, the user's past fitness goals or content on their fitness device (e.g., calendar appointments, text messages with friends or trainer, use of fitness app 107, and third-party fitness app use) about upcoming workouts for the day can be used to infer their caloric expenditure for the day. For example, the user does a run every Monday after work from 7-8 PM that burns 500 calories on average in 30 minutes. In this example, the user would be notified by their fitness device with a notification stating, e.g., “You can cover the calories from this bag of chips with 7 minutes of your run this evening.” If the fitness goal is time-based, the user can be recommended (or reminded) to add 7 minutes to their target workout time. This adjustment can also be performed automatically if the user has opted in. The user's learned routine can be leveraged.

In an example case, a user runs on Mondays and plays tennis on Saturdays regularly. If they wanted to eat that bag of chips on Saturday, their notification would be in the context of tennis instead of running, because the personalized PACE generator 104 has determined their preferred sport and their typical intensity for their exercise on that day from the fitness data provided by fitness application 107. In this example case where the user currently burns 1000 calories on average in 30 minutes playing tennis, they would receive a notification (e.g., displayed and/or audio) on their fitness device, stating, e.g., “you can burn the calories from this bag of chips by playing 3 additional minutes of tennis this evening.”

In other embodiments, an alternative workout can be suggested for that day. For example, if the user's routine activities include tennis and running, a tennis workout instead of a running workout on a “running day” (e.g. Monday) can be recommended to the user, even though Monday is not their “tennis day” (e.g. Saturday), if they burn more calories per minute during a tennis session than a running session. In some embodiments, a preemptive suggestion can be made based on calendar events, text messages or emails of the user. For example, if the user mentions dinner plans at a fast food restaurant in their text messages, the personalized PACE generator 104 can look up the average number of calories in a meal at that restaurant (e.g., through an Internet search), or past user behavior at similar restaurants if available, and recommend a workout duration and exercise type for the user.

In some embodiments, recommendations can be made to the user to cover caloric consumption from previous meals. For example, if a user ate a bag of chips that was not in their nutrition plan for the day before and the user did not move enough to account for that bag of chips, the user can be prompted to work out for a specific duration to cover for that bag of chips the following day.

In some embodiments, the fitness application 107 uses and the fitness and workout history of the user to recommend workouts to the user. For example, if the user normally engages in 30-minutes of HIIT training on fitness application 107 on Monday and would like to burn an additional 90 calories from the bag of chips, they can get a recommendation for an appropriate workout on the physical fitness app 107 to reach that goal, such as, e.g., a more intense 30-minute HIIT session, or a 45-minute HIIT session with a similar intensity to reach that goal.

In some embodiments, recommendations specific to the user can also be made, such as a recommendation of exercise calories, for example: “You would need to run an extra 90 calories on your run tonight to burn off the calories from these chips. Add to tonight's workout?” In some embodiments, an example recommendation of exercise minutes could be, e.g., “You would need to run an extra 7 minutes on your run tonight to burn off the calories from these chips. Add to tonight's workout?” If the user has opted in, when they eat the bag of chips then 90 calories is automatically added to their physical fitness goal for the day. In some embodiments, a reminder to train (time-based), may provide the recommendation, e.g.: “You normally run on Mondays. Run for at least 39 minutes this evening to stay on track with today's eating goals,” assuming the user ate the chips in addition to their usual diet.

In some embodiments, the user can be allowed to opt-in for only certain types of foods. For example, a suggestion can be made for foods that are determined to have an unfavorable macronutrient split (e.g., higher in fats and/or carbs, “junk food”, “cheat meals” determined by the user like pizza, dessert or snacks) or foods that are not part of the user's regular diet/caloric budget.

In some embodiments, a person or location or exercise information in a calendar or text message can be used to estimate upcoming calorie consumption for PACE. For example, on average a tennis game with person “A” might be more intense than a tennis game with person “B”, or a swim at pool “X” might usually be more intense than a swim at pool “Y”.

In some embodiments, other methods of finding nutritional information can be used other than image capture using a camera, provided there is a good way of identifying the calories in a meal. For example, computer vision approaches and a database and/or recipe can be used instead of the nutritional facts label. It could be done such that workout durations and “end goals” of a workout are determined by the nutrient that is consumed, such as, e.g., a workout to burn off an extra 90 calories' to account for the bag of chips. In some embodiments, the user can manually enter (e.g., through a graphical user interface) what they are consuming to get the same type of contextual feedback. A similar approach could be taken with calories instead of exercise minutes to keep individuals on track if their personal goals are calorie-based. This may enable other new features. For example, it can enable suggestions for portioning. If a user typically works out in the morning but misses their exercise for the day and has thus moved less, the user can be given a recommended portion size for a certain food to stay on track with their calorie consumption goals. This could be done without a camera and can be done with manual data entry (e.g., meal logging/calorie tracking and/or exercise plan).

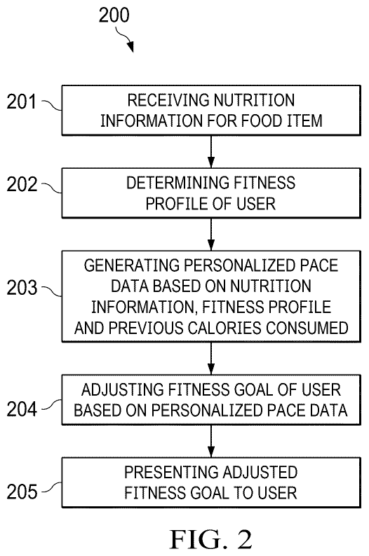

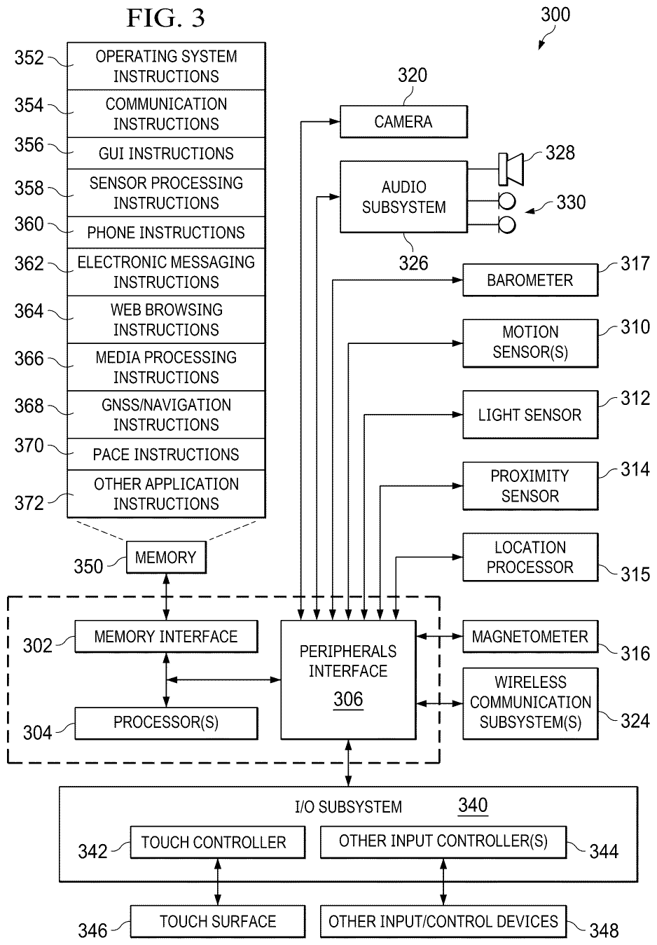

FIG. 2 is a flow diagram of process 200 implemented by a fitness device that generates and presents personalized PACE feedback based on a user's fitness profile, according to one or more embodiments. Process 200 can be implemented using, for example, the fitness device architecture 300 described in reference to FIG. 3

Process 200 includes: receiving nutrition information for a food item to be consumed by a user of the fitness device (201); determining a fitness profile of the user, including physical activity data of the user and a fitness goal of the user (202); generating physical activity calorie equivalent data based on the nutrition information, the physical fitness profile and previous calories consumed by the user (203); adjusting the fitness goal of the user based on the physical activity calorie equivalent data (204); and presenting the adjusted fitness goal to the user (205). Each of these steps was previously described in reference to FIG. 1.

FIG. 3 is a block diagram of an architecture 300 for a fitness device that generates and presents personalized PACE feedback based on a user's physical fitness profile, as described in reference to FIGS. 1-2. Architecture 300 can include memory interface 302, one or more hardware data processors, image processors and/or processors 304 and peripherals interface 306. Memory interface 302, one or more processors 304 and/or peripherals interface 306 can be separate components or can be integrated in one or more integrated circuits. System 300 can be included in any suitable electronic device coupled to a display, including but not limited to desktop computers, notebook computers, tablet computers, and the like.

Sensors, devices, and subsystems can be coupled to peripherals interface 306 to provide multiple functionalities. For example, one or more motion sensors 310, light sensor 312 and proximity sensor 314 can be coupled to peripherals interface 306 to facilitate motion sensing (e.g., acceleration, rotation rates), lighting and proximity functions of the wearable device. Location processor 315 can be connected to peripherals interface 306 to provide geo-positioning. In some implementations, location processor 315 can be a GNSS receiver, such as the Global Positioning System (GPS) receiver. Electronic magnetometer 316 (e.g., an integrated circuit chip) can also be connected to peripherals interface 306 to provide data that can be used to determine the direction of magnetic North. Electronic magnetometer 316 can provide data to an electronic compass application. Motion sensor(s) 310 can include one or more accelerometers and/or gyros configured to determine change of speed and direction of movement. Barometer 317 can be configured to measure atmospheric pressure (e.g., pressure change inside a vehicle). Camera 320 can be a still or video camera of any suitable type that has the ability capture data from nutritional fact labels or search for barcodes. In some embodiments, a bio signal sensor (not shown) can be included in architecture 300 and can be one or more of a PPG sensor, an electroencephalogram (EEG) sensor, an electrocardiogram (ECG) sensor, an electromyogram (EMG) sensor, a mechanomyogram (MMG) sensor (e.g., piezoresistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 324, which can include radio frequency (RF) receivers and transmitters (or transceivers) and/or optical (e.g., infrared) receivers and transmitters. The specific design and implementation of the communication subsystem 324 can depend on the communication network(s) over which a mobile device is intended to operate. For example, architecture 300 can include communication subsystems 324 designed to operate over a GSM network, a GPRS network, an EDGE network, a WiFi™ network and a Bluetooth™ network. In particular, the wireless communication subsystems 324 can include hosting protocols, such that the crash device can be configured as a base station for other wireless devices.

Audio subsystem 326 can be coupled to a speaker 328 and a microphone 330 to facilitate voice-enabled functions, such as voice recognition, voice replication, digital recording and telephony functions. Audio subsystem 326 can be configured to receive voice commands from the user.

I/O subsystem 340 can include touch surface controller 342 and/or other input controller(s) 344. Touch surface controller 342 can be coupled to a touch surface 346. Touch surface 346 and touch surface controller 342 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 346. Touch surface 346 can include, for example, a touch screen or the digital crown of a smart watch. I/O subsystem 340 can include a haptic engine or device for providing haptic feedback (e.g., vibration) in response to commands from processor 304. In an embodiment, touch surface 346 can be a pressure-sensitive surface.

Other input controller(s) 344 can be coupled to other input/control devices 348, such as one or more buttons, rocker switches, thumbwheel, infrared port, and USB port. The one or more buttons (not shown) can include an up/down button for volume control of speaker 328 and/or microphone 330. Touch surface 346 or other controllers 344 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 546; and a pressing of the button for a second duration that is longer than the first duration may turn power to the mobile device on or off. The user may be able to customize a functionality of one or more of the buttons. The touch surface 346 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 302 can be coupled to memory 350. Memory 350 can include high-speed random-access memory and/or non-volatile memory, such as one or more magnetic disk storage devices, one or more optical storage devices and/or flash memory (e.g., NAND, NOR). Memory 350 can store operating system 352, such as the iOS operating system developed by Apple Inc. of Cupertino, California. Operating system 352 may include instructions for handling basic system services and for performing hardware dependent tasks. In some implementations, operating system 352 can include a kernel (e.g., UNIX kernel).

Memory 350 may also store communication instructions 354 to facilitate communicating with one or more additional devices, one or more computers and/or one or more servers, such as, for example, instructions for implementing a software stack for wired or wireless communications with other devices. Memory 350 may include graphical user interface instructions 356 to facilitate graphic user interface processing; sensor processing instructions 358 to facilitate sensor-related processing and functions; phone instructions 360 to facilitate phone-related processes and functions; electronic messaging instructions 362 to facilitate electronic-messaging related processes and functions; web browsing instructions 364 to facilitate web browsing-related processes and functions; media processing instructions 366 to facilitate media processing-related processes and functions; GNSS/Location instructions 368 to facilitate generic GNSS and location-related processes and instructions; and PACE instructions 370 that implement the processes described in reference to FIGS. 1-2. Memory 350 further includes other application instructions 372 including but not limited to instructions for applications (e.g., fitness or health monitoring applications) that use the features and processed described in reference to FIGS. 1-2.

Each of the above identified instructions and applications can correspond to a set of instructions for performing one or more functions described above. These instructions need not be implemented as separate software programs, procedures, or modules. Memory 350 can include additional instructions or fewer instructions. Furthermore, various functions of the mobile device may be implemented in hardware and/or in software, including in one or more signal processing and/or application specific integrated circuits.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

What is claimed is:

1. A method comprising:
receiving, with at least one processor of a fitness device, nutrition information for a food item to be consumed by a user of the fitness device;
determining, with the at least one processor, a fitness profile of the user, including activity data of the user and a fitness goal of the user;
generating, with the at least one processor, personalized physical activity calorie equivalent data based on the nutrition information, the fitness profile and previous calories consumed by the user;
adjusting, with the at least one processor, the exercise goal of the user based on the physical activity calorie equivalent data; and
presenting, with the at least one processor, the adjusted exercise goal to the user.

2. The method of claim 1, wherein the fitness profile further includes demographic data for the user.

3. The method of claim 1, wherein the fitness profile further includes historical workout pattern data for the user.

4. The method of claim 1, wherein the fitness profile further includes a preferred exercise type of the user.

5. The method of claim 1, wherein the fitness profile further includes an exercise habit of the user.

6. The method of claim 1, wherein the nutrition information for the food item to be consumed by a user is captured from a nutritional fact label for the food item by a camera of the fitness device.

7. The method of claim 1, wherein the activity data of the user is determined from sensor data provided by sensors of the fitness device.

8. The method of claim 1, wherein the fitness profile is provided by a fitness application running on the fitness device.

9. The method of claim 1, wherein the adjusted exercise goal includes an exercise type for the user to engage in to burn off calories in the food item, and wherein the exercise type is the preferred exercise type in the physical fitness profile.

10. The method of claim 9, wherein the adjusted exercise goal includes an amount of time for the user to engage in the exercise type to burn off the calories in the food item.

11. A fitness device comprising:
a display;
one or more processors;
memory storing instructions that when executed by the one or more processors, causes the one or more processors to perform operations comprising:
receiving nutrition information for a food item to be consumed by a user of the fitness device;
determining a fitness profile of the user, including activity data of the user and a fitness goal of the user;
generating physical activity calorie equivalent data based on the nutrition information, the physical fitness profile and previous calories consumed by the user;
adjusting the exercise goal of the user based on the physical activity calorie equivalent data; and
presenting on the display the adjusted exercise goal to the user.

12. The fitness device of claim 11, wherein the fitness profile further includes demographic data for the user.

13. The fitness device of claim 11, wherein the fitness profile further includes historical workout pattern data for the user.

14. The fitness device of claim 11, wherein the fitness profile further includes a preferred exercise type of the user.

15. The fitness device of claim 11, wherein the fitness profile further includes an exercise habit of the user.

16. The fitness device of claim 11, wherein the nutrition information for the food item to be consumed by a user is captured from a nutritional fact label for the food item by a camera of the fitness device.

17. The fitness device of claim 11, wherein the activity data of the user is determined from sensor data provided by sensors of the fitness device.

18. The fitness device of claim 11, wherein the fitness profile is provided by a fitness application running on the fitness device.

19. The fitness device of claim 11, wherein the adjusted exercise goal includes an exercise type for the user to engage in to burn off calories in the food item, and wherein the exercise type is the preferred exercise type in the fitness profile.

20. The fitness device of claim 19, wherein the adjusted exercise goal includes an amount of time for the user to engage in the exercise type to burn off the calories in the food item.
